(19) United States
(12) Patent Application Publication
(10) Pub. No.: US 2026/0110676 A1
(43) Pub. Date: Apr. 23, 2026

(54) POLAR COORDINATE-BASED METHOD FOR EVALUATING NUTRIENT EFFICIENCY OF CROP

(71) Applicant: Chinese Academy of Agricultural Sciences Institute of Crop Science, Beijing (CN)

(72) Inventors: Zheng Liu, Beijing (CN); Congfeng Li, Beijing (CN); Baoyuan Zhou, Beijing (CN); Zaisong Ding, Beijing (CN); Xinbing Wang, Beijing (CN)

(21) Appl. No.: 19/319,776

(22) Filed: Sep. 5, 2025

(30) Foreign Application Priority Data
Oct. 23, 2024 (CN) 2024114849440

(51) Int. Cl. G01N 33/00; G06Q 50/02

(52) U.S. Cl. CPC G01N 33/0098; G06Q 50/02

(57) ABSTRACT

A polar coordinate-based method for evaluating a nutrient efficiency of a crop includes the following steps: establishing a polar coordinate system with a yield of a variety to be tested in a nutrient-deficient plot as a horizontal coordinate, a yield of the variety to be tested in a normal plot as a vertical coordinate, and an average yield of multiple control varieties as an origin; obtaining a polar coordinate of the variety to be tested based on the polar coordinate system established; calculating a projection difference based on polar coordinate data of the variety to be tested; and evaluating a nutrient efficiency of the variety to be tested through the projection difference. The polar coordinate-based method can realize accurate and rapid evaluation of the nutrient efficiency of the crop without chemical component detection and has advantages of low cost, easy operation, and comparable results in different plots.

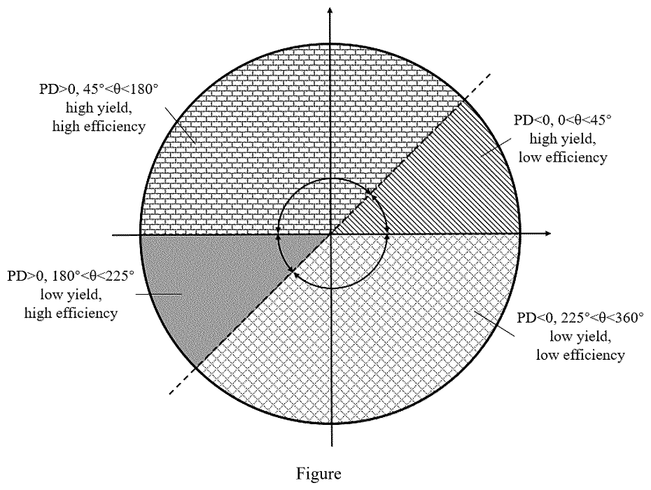

### CROSS-REFERENCE TO RELATED APPLICATION

This application claims priority to Chinese Patent Application No. 202411484944.0, filed on Oct. 23, 2024, which is herein incorporated by reference in its entirety.

### TECHNICAL FIELD

The disclosure relates to the field of crop cultivation technologies, and more particularly to a polar coordinate-based method for evaluating a nutrient efficiency of a crop.

### BACKGROUND

Application of synthetic chemical fertilizers has greatly increased crop yield and ensured global food security. Breeding crop varieties with high nutrient efficiency is an important measure to improve nutrient utilization efficiencies of crops, which is inseparable from evaluation technology and calculation method of a nutrient efficiency of a crop. Currently, indicators such as fertilizer partial productivity, fertilizer agronomic efficiency, fertilizer recovery rate, and nutrient physiological utilization rate are often used to measure the nutrient efficiency of the crop. However, these indicators have the following defects, respectively.

### (1) Fertilizer Partial Productivity

$$fertilizer partial productivity = crop yield / fertilizer application amount$$

Apparently, the fertilizer partial productivity is significantly influenced by soil factors. Nutrient supply capacity (nutrient effectiveness) varies significantly across different regions and soils, resulting in poor reliability of cross-regional and horizontal comparisons. Specifically, in areas with fertile soil, the nutrient efficiency of the crop is underestimated, whereas in areas with poor soil conditions, the nutrient efficiency of the crop is overestimated. Therefore, the fertilizer partial productivity is limited by the soil and climate conditions at a test plot, and comparability of results at different plots is poor.

### (2) Fertilizer Agronomic Efficiency

$$fertilizer agronomic efficiency = ( yield in fertilized plots - yield in unfertilized plots ) / fertilizer application amount$$

The fertilizer agronomic efficiency introduces the yield in unfertilized plots as a control, which to some extent avoids influence of the soil factors but ignores genotype differences in the crop response to the nutrient effectiveness, resulting in a lack of reliable reference for comparison across different plots and varieties.

### (3) Fertilizer Recovery Rate and Nutrient Physiological Utilization Rate

$$fertilizer recovery rate = ( nutrient accumulation of crop harvesting organs in fertilized plots - nutrients accumulation of crop harvesting organs in unfertilized plots ) / f ertilizer application amount$$

$$nutrient physiological utilization rate = biomass of crop harvesting organs / crop nutrient accumulation$$

The fertilizer recovery rate and the nutrient physiological utilization rate measure the nutrient efficiency of the crop from perspectives of nutrient absorption and assimilation, respectively. However, their calculation methods rely on chemical analysis, and samples must undergo multiple processing steps such as drying, crushing, digestion, and chemical stoichiometry (such as Kjeldahl method for nitrogen determination, flame combustion method for potassium measurement, and vanadium molybdenum yellow colorimetric method for phosphorus measurement) to obtain nutrient content data, which is costly and time-consuming.

### SUMMARY

Based on the aforementioned technical problems, the disclosure, based on a polar coordinate and vector analysis, provides a polar coordinate-based method for evaluating a nutrient efficiency of a crop, to thereby realize accurate and rapid evaluation of the nutrient efficiency of the crop without chemical component detection. The polar coordinate-based method has advantages of low cost, easy operation, and comparable results in different plots.

Specific technical solutions provided by the disclosure are as follows.

The polar coordinate-based method for evaluating the nutrient efficiency of the crop provided by the disclosure includes the following steps:
establishing a polar coordinate system with a yield of a variety to be tested in a nutrient-deficient plot as a horizontal coordinate, a yield of the variety to be tested in a normal plot as a vertical coordinate, and an average yield of multiple control varieties as an origin;
obtaining a polar coordinate of the variety to be tested based on the polar coordinate system established; and
calculating a projection difference based on polar coordinate data of the variety to be tested, and evaluating a nutrient efficiency of the variety to be tested through the projection difference.

In an embodiment, the polar coordinate-based method further includes selecting a target variety as a potential breeding variety with desired nutrient efficiency after the evaluating a nutrient efficiency of the variety to be tested through the projection difference, and then planting the target variety.

In an embodiment, the polar coordinate data of the variety to be tested includes a feature vector with a radius of r and a polar angle of θ₀.

In an embodiment, a polar coordinate of a j-th variety to be tested is specifically as follows:

$${r}_{j} = \sqrt{{( {Tj}_{N 0} - \frac{{\sum }_{i = 1}^{n} C {i}_{N 0}}{n} )}^{2} + {( {Tj}_{N 225} - \frac{{\sum }_{i = 1}^{n} {Ci}_{N 225}}{n} )}^{2}}$$

$${θ}_{j} = {tan}^{- 1} \frac{{Tj}_{N 225} - \frac{{\sum }_{i = 1}^{n} {Ci}_{N 225}}{n}}{{Tj}_{N 0} - \frac{{\sum }_{i = 1}^{n} {Ci}_{N 0}}{n}}$$

where rⱼ is a radius of the j-th variety to be tested, θⱼ is a polar angel of the j-th variety to be tested, Tj_{N0} represents a yield of the j-th variety to be tested in the nutrient-deficient plot, Tj_{N225} represents a yield of the j-th variety to be tested in the normal plot, Ci_{N0} represents a yield of an i-th control variety in the nutrient-deficient plot, Ci_{N225} represents a yield of the i-th control variety in the normal plot, and n represents a number of the multiple control varieties.

In an embodiment, a calculation formula for the projection difference is specifically as follows:

$$P D = ( sin {θ}_{0} - cos {θ}_{0} ) * r$$

where PD represents the projection difference, θ₀ represents the polar angle in the polar coordinate data of the variety to be tested, θ₀ is equal to or larger than 0 degrees (°) and less than 360°, and r represents the radius in the polar coordinate data of the variety to be tested.

In an embodiment, criteria for evaluating a nutrient efficiency of the variety to be tested through the projection difference are as follows:
when PD<0, the nutrient efficiency of the variety to be tested is lower than that of the multiple control varieties;
when PD=0, the nutrient efficiency of the variety to be tested is equal to that of the multiple control varieties;
when PD>0, the nutrient efficiency of the variety to be tested is higher than that of the multiple control varieties.

In an embodiment, the polar coordinate-based method further includes evaluating a yield potential of the variety to be tested by using the polar angle in the polar coordinate data of the variety to be tested as an index.

In an embodiment, criteria for the evaluating a yield potential of the variety to be tested by using the polar angle are as follows:
when 0<θ₀<180°, the yield potential of the variety to be tested is higher than that of the multiple control varieties;
when 180°<θ₀<360°, the yield potential of the variety to be tested is lower than that of the multiple control varieties.

In an embodiment, the polar coordinate-based method further includes comprehensively evaluating the nutrient efficiency and the yield potential of the variety to be tested through the projection difference and the polar angle in the polar coordinate data of the variety to be tested.

In an embodiment, criteria for the comprehensively evaluating the nutrient efficiency and the yield potential of the variety to be tested are as follows:
when PD>0 and 45°<θ₀<180°, the nutrient efficiency and the yield potential of the variety to be tested are both higher than those of the multiple control varieties;
when PD>0 and 180°<θ₀<225°, the nutrient efficiency of the variety to be tested is higher than that of the multiple control varieties, but the yield potential of the variety to be tested is lower than that of the multiple control varieties;
when PD<0 and 0<θ₀<45°, the nutrient efficiency of the variety to be tested is lower than that of the multiple control varieties, but the yield potential of the variety to be tested is higher than that of the multiple control varieties;
when PD<0 and 225°<θ₀<360°, the nutrient efficiency and the yield potential of the variety to be tested are both lower than those of the multiple control varieties.

In an embodiment, the nutrient efficiency refers to a utilization efficiency of nitrogen, phosphorus, and potassium by the crop.

In an embodiment, the crop includes but is not limited to corn, wheat, rice, soybean, fruits, and vegetables.

Compared with the related art, the disclosure has the following beneficial effects.

The disclosure, based on the polar coordinate and the vector analysis, establishes the polar coordinate-based method for evaluating the nutrient efficiency of the crop without relying on the chemical component detection from a perspective of the crop response to fertilizer application. The polar coordinate-based method has the advantages of low cost, easy operation, and comparable results in different plots.

The polar coordinate-based method provided in the disclosure can be applied to evaluate the nutrient efficiency of the crop of macronutrient elements such as nitrogen, phosphorus, and potassium, and is applicable to crops including but not limited to the corn, the rice, the wheat, the fruits and the vegetables.

### BRIEF DESCRIPTION OF DRAWING

FIGURE illustrates a polar coordinate-based method for evaluating a nutrient efficiency and a yield potential of a crop according to an embodiment of the disclosure.

### DETAILED DESCRIPTION OF EMBODIMENTS

Illustrative embodiments of the disclosure will be described in more detail as follows. Although the illustrative embodiments of the disclosure are described as follows, it should be understood that the disclosure can be implemented in various forms and should not be limited by embodiments described herein. On a contrary, these embodiments are provided so that the disclosure can be more thoroughly understood, and a scope of protection of the disclosure can be fully conveyed to those skilled in the art. It should be noted that the embodiments in the disclosure and the features in the embodiments can be combined with each other without conflict. The disclosure will be described in detail with reference to the embodiments.

In related art, indicators such as fertilizer partial productivity, fertilizer agronomic efficiency, fertilizer recovery rate, and nutrient physiological utilization rate are often used to measure a nutrient efficiency of a crop. However, these indicators have the following defects, respectively.

The fertilizer partial productivity is limited by soil and climate conditions at a test plot, and comparability of results at different plots is poor. The fertilizer agronomic efficiency ignores genotype differences in the crop response to nutrient effectiveness, resulting in a lack of reliable reference for comparison across different plots and varieties. Calculation methods of the fertilizer recovery rate and the nutrient physiological utilization rate rely on chemical analysis, and samples must undergo multiple processing steps such as drying, crushing, digestion, and chemical stoichiometry to obtain nutrient content data, which is costly and time-consuming.

Based on this, the disclosure provides a polar coordinate-based method for evaluating the nutrient efficiency of the crop, including the following steps 1 through 3.
Step 1, a polar coordinate system is established with a yield of a variety to be tested in a nutrient-deficient plot as a horizontal coordinate, a yield of the variety to be tested in a normal plot as a vertical coordinate, and an average yield of multiple control varieties as an origin.
Step 2, a polar coordinate of the variety to be tested is obtained based on the polar coordinate system established.
Step 3, a projection difference is calculated based on polar coordinate data of the variety to be tested. A nutrient efficiency of the variety to be tested is evaluated through the projection difference.

By using the polar coordinate-based method provided by the disclosure, the nutrient efficiency of the crop can be accurately and rapidly evaluated without chemical component detection. The polar coordinate-based method has advantages of low cost, easy operation, and comparable results in different plots.

The disclosure will be described in detail with reference to specific embodiments.

Evaluating the nutrient efficiency of the crop based on the polar coordinate includes the following steps.
(1) Taking nitrogen nutrient efficiency of corn as an example, a test is conducted on a nitrogen-deficient plot without applying nitrogen fertilizer for more than five consecutive years and a normal plot with an application amount of the nitrogen fertilizer of 225 kilograms of nitrogen per hectare (kg N/ha) (i.e., an internationally recognized upper limit of safe application). Test materials are m varieties to be tested (marked as T1, T2, T3 . . . . Tm) and n control varieties (marked as C1, C2, C3 . . . . Cn). Other agronomic measures and links, such as the control varieties, amount of the control varieties, tillage methods, planting area, planting density, phosphorus and potassium fertilizer application amount, pest and disease management, weed control, harvesting, and storage refer to “Approval Measures for Major Crop Varieties” revised in 2022.
(2) In the nitrogen-deficient plot, yields of the m varieties to be tested are respectively T1_{N0}, T2_{N0}, T3_{N0} . . . Tm_{N0}, and yields of the n control varieties are respectively C1_{N0}, C2_{N0}, C3_{N0} . . . Cn_{N0}. In the normal plot, yields of the m varieties to be tested are respectively T1_{N225}, T2_{N225}, T3_{N225} . . . Tm_{N225}, and yields of the n control varieties are respectively C1_{N225}, C2_{N225}, C3_{N225} . . . Cn_{N225}.
(3) The polar coordinate system is established with the yields of the m varieties to be tested in the nitrogen-deficient plot as horizontal coordinates (x-axis), the yields of the m varieties to be tested in the normal plot as vertical coordinates (y-axis), and an average yield of the n control varieties as the origin (also referred to as a pole). A polar coordinate of a j-th variety to be tested is specifically as follows:

$${r}_{j} = \sqrt{{( {Tj}_{N 0} - \frac{{\sum }_{i = 1}^{n} C {i}_{N 0}}{n} )}^{2} + {( {Tj}_{N 225} - \frac{{\sum }_{i = 1}^{n} {Ci}_{N 225}}{n} )}^{2}}$$

$${θ}_{j} = {tan}^{- 1} \frac{{Tj}_{N 225} - \frac{{\sum }_{i = 1}^{n} {Ci}_{N 225}}{n}}{{Tj}_{N 0} - \frac{{\sum }_{i = 1}^{n} {Ci}_{N 0}}{n}} .$$

At this time, each of the m varieties to be tested has a feature vector with a radius of r and a polar angle of θ₀, i represents an i-th control variety.

The nutrient efficiency is represented by a yield increase from the nitrogen-deficient plot to the normal plot. When a yield increase of the variety to be tested is greater than that of a control variety, a nutrient efficiency of the variety to be tested is higher than that of the control variety. When the increase of the variety to be tested is less than that of the control variety, the nutrient efficiency of the variety to be tested is lower than that of the control variety.

The projection difference (PD) can be used to measure the nutrient efficiency of the crop, with a calculation method therefor specifically as follows:

$$PD = ( sin {θ}_{0} - cos {θ}_{0} ) * r$$

where θ₀ represents the polar angle in the polar coordinate of the variety to be tested, r represents the radius in the polar coordinate of the variety to be tested, θ₀ is in a range of [0, 360)°, namely, 0≤θ₀<360°.
(4) The nutrient efficiency and a yield potential of the crop are evaluated, including sub-steps 1 through 3, criteria are illustrated in FIGURE.

Sub-step 1, the nutrient efficiency of the crop is evaluated through the PD according to the following criteria:
when PD<0, the nutrient efficiency of the variety to be tested is lower than that of the multiple control varieties;
when PD=0, the nutrient efficiency of the variety to be tested is equal to that of the multiple control varieties;
when PD>0, the nutrient efficiency of the variety to be tested is higher than that of the multiple control varieties.

Sub-step 2, the yield potential of the crop is evaluated through the polar angle θ₀ according to the following criteria:
when 0<θ₀<180°, the yield potential of the variety to be tested is higher than that of the multiple control varieties;
when 180°<θ₀<360°, the yield potential of the variety to be tested is lower than that of the multiple control varieties.

The nutrient efficiency and the yield potential of the crop are comprehensively evaluated through a combination of the PD and the θ₀ according to the following criteria:
when PD>0 and 45°<θ₀<180°, the nutrient efficiency and the yield potential of the variety to be tested are both higher than those of the multiple control varieties;
when PD>0 and 180°<θ₀<225°, the nutrient efficiency of the variety to be tested is higher than that of the multiple control varieties, but the yield potential of the variety to be tested is lower than that of the multiple control varieties;
when PD<0 and 0<θ₀<45°, the nutrient efficiency of the variety to be tested is lower than that of the multiple control varieties, but the yield potential of the variety to be tested is higher than that of the multiple control varieties;
when PD<0 and 225°<θ₀<360°, the nutrient efficiency and the yield potential of the variety to be tested are both lower than those of the multiple control varieties.

The polar coordinate-based method provided by the disclosure can be applied to evaluate the nutrient efficiency of the crop of macronutrient elements such as nitrogen, phosphorus, and potassium, and is applicable to crops including but not limited to the corn, rice, wheat, fruits, and the vegetables.

The disclosure will be described with reference to the specific embodiments.

Test design includes 5 varieties to be tested and 1 control variety, both of which are set with two groups of nitrogen fertilizer application amounts of 0 and 225 kg N/ha. The test has been conducted at the same plot for nine consecutive years. At a physiological maturity stage (harvest stage), a grain yield, a grain nitrogen concentration, a straw biomass, and a straw nitrogen concentration of the corn are measured to obtain measurement results. Relevant evaluation indicators such as nitrogen partial productivity, agronomic nitrogen efficiency, nitrogen recovery rate, nitrogen physiological utilization rate, the PD, and θ₀ are calculated to obtain calculation results. Situation of the 5 varieties to be tested and the 1 control variety is shown in table 1. The measurement results in the harvest stage are shown in table 2. The calculation results of the relevant evaluation indicators are shown in table 3.

TABLE 1
variety number, variety name, and variety breeding year

| Variety number | Variety name | Variety breeding year |
|---|---|---|
| T1 | Baimaya | 1950s |
| T2 | Danyu13 | 1970s |
| T3 | Shendan7 | 1980s |
| T4 | Xianyu335 | 2000s |
| T5 | Denghai605 | 2010s |
| C1 | Zhengdan958 | 1990s |

In the table 1, the corn variety of Baimaya is a local variety bred from an open-pollinated variety and formed through long-term natural selection and domestication by farmers after introduction in mid-20th century. The corn variety of Danyu13, with a registration number of GS03003-1989, is developed through crossbreeding Mo17Ht as a female parent and E28 as a male parent by Dandong Academy of Agricultural Sciences, Liaoning Province in 1970. The corn variety of Shendan7, with the registration number of GS03001-1990, is developed through crossbreeding 5003 as the female parent and E28 as the male parent by Shenyang Academy of Agricultural Sciences, Liaoning Province in 1982. The corn variety of Xianyu335, with the registration number of GS2004017/GS2006026, is developed through crossbreeding PH6WC as the female parent and PH4CV as the male parent by Tieling Pioneer Seed Research Company Limited (Co., Ltd.) in 2000. The corn variety of Denghai605, with the registration number of GS2010009, is developed through crossbreeding DH351 as the female parent and DH382 as the male parent by Shandong Denghai Seeds Co., Ltd. in 2005. The corn variety of Zhengdan958, with the registration number of GS20000009, is developed through crossbreeding Zheng58 as the female parent and Chang7-2 as the male parent by Institute of Grain Crops, Henan Academy of Agricultural Sciences in 1996.

TABLE 2
grain yield, grain nitrogen concentration, straw biomass, and
straw nitrogen concentration at physiological maturity stage

| Nitrogen fertilizer | | Grain yield (ton | Grain | | Straw |
|---|---|---|---|---|---|
| application amount | | per hectare, | nitrogen | Straw | nitrogen |
| (kilogram per hectare, | Variety | abbreviated | concentration | biomass | concentration |
| abbreviated as kg/ha) | number | as t/ha) | (%) | (t/ha) | (%) |
| 0 | T1 | 4.5 | 1.5 | 5.3 | 1.0 |
| | T2 | 4.7 | 1.5 | 4.9 | 1.1 |
| | T3 | 5.2 | 1.5 | 5.4 | 1.1 |
| | T4 | 6.1 | 1.6 | 5.2 | 1.2 |
| | T5 | 5.6 | 1.7 | 5.2 | 1.2 |
| | C1 | 5.3 | 1.6 | 5.1 | 1.2 |
| 225 | T1 | 6.6 | 1.7 | 7.2 | 1.4 |
| | T2 | 7.5 | 1.8 | 7.7 | 1.5 |
| | T3 | 8.1 | 1.8 | 7.6 | 1.6 |
| | T4 | 11.4 | 2.1 | 10.3 | 1.8 |
| | T5 | 13.9 | 2.2 | 11.4 | 1.9 |
| | C1 | 10.8 | 2.1 | 9.9 | 1.8 |

TABLE 3
nitrogen partial productivity, agronomic nitrogen efficiency, nitrogen
recovery rate, nitrogen physiological utilization rate, PD, and θ₀

| | nitrogen partial | agronomic | | nitrogen | | |
|---|---|---|---|---|---|---|
| | productivity | nitrogen | nitrogen | physiological | | |
| Variety | (kilogram per kilogram, | efficiency | recovery | utilization | | |
| number | abbreviated as kg/kg) | (kg/kg) | rate (%) | rate (%) | PD | θ₀ |
| T1 | 29.3 | 9.0 | 53.6 | 25.1 | −3.4 | 258.05 |
| T2 | 33.3 | 12.4 | 55.3 | 26.7 | −2.7 | 258.69 |
| T3 | 36.0 | 12.9 | 61.1 | 29.5 | −2.6 | 264.29 |
| T4 | 50.7 | 23.6 | 71.1 | 35.4 | −0.2 | 36.87 |
| T5 | 61.8 | 36.9 | 70.0 | 36.0 | 2.8 | 84.29 |
| C1 | 48.0 | 24.4 | 64.9 | 34.2 | — | — |

Correlation analysis shows that Pearson coefficients between the PD and the nitrogen partial productivity, the agronomic nitrogen efficiency, the nitrogen recovery rate, and the nitrogen physiological utilization efficiency are 0.9887, 0.9999, 0.8570, and 0.9042, respectively, and all the Pearson coefficients reach highly significant levels. This indicates that the PD can be well used to evaluate the nutrient efficiency of the crop. Based on the criteria provided in the disclosure, nutrient efficiencies and yield potentials of T1, T2, and T3 are lower than those of the control variety. A nutrient efficiency of T4 is lower than that of the control variety, but a yield potential of T4 is higher than that of the control variety. A nutrient efficiency and a yield potential of T5 are higher than those of the control variety, so T5 can be used as a potential breeding variety with high nutrient efficiency.

Lastly, it should be noted that, the aforementioned embodiments are merely used to illustrate the disclosure, rather than limit the scope of protection of the disclosure. Although the disclosure has been described in detail through the aforementioned embodiments, it should be understood by those skilled in the art that various changes can be made in form and detail without departing from the scope of protection of the disclosure specified in the claims.

What is claimed is:

1. A polar coordinate-based method for evaluating a nutrient efficiency of a crop, comprising the following steps:
establishing a polar coordinate system with a yield of a variety to be tested in a nutrient-deficient plot as a horizontal coordinate, a yield of the variety to be tested in a normal plot as a vertical coordinate, and an average yield of a plurality of control varieties as an origin;
obtaining a polar coordinate of the variety to be tested based on the polar coordinate system established; and
calculating a projection difference based on polar coordinate data of the variety to be tested, and evaluating a nutrient efficiency of the variety to be tested through the projection difference;
wherein the polar coordinate data of the variety to be tested comprises a feature vector with a radius of r and a polar angle of θ₀, and a polar coordinate of a j-th variety to be tested is specifically as follows:
$${r}_{j} = \sqrt{{( {Tj}_{N 0} - \frac{{\sum }_{i = 1}^{n} C {i}_{N 0}}{n} )}^{2} + {( {Tj}_{N 225} - \frac{{\sum }_{i = 1}^{n} {Ci}_{N 225}}{n} )}^{2}}$$
$${θ}_{j} = {tan}^{- 1} \frac{{Tj}_{N 225} - \frac{{\sum }_{i = 1}^{n} {Ci}_{N 225}}{n}}{{Tj}_{N 0} - \frac{{\sum }_{i = 1}^{n} {Ci}_{N 0}}{n}}$$
where rⱼ is a radius of the j-th variety to be tested, θⱼ is a polar angel of the j-th variety to be tested, Tj_{N0} represents a yield of the j-th variety to be tested in the nutrient-deficient plot, Tj_{N225} represents a yield of the j-th variety to be tested in the normal plot, Ci_{N0} represents a yield of an i-th control variety in the nutrient-deficient plot, Ci_{N225} represents a yield of the i-th control variety in the normal plot, and n represents a number of the plurality of control varieties; and
wherein a calculation formula for the projection difference is specifically as follows:
$$PD = ( sin {θ}_{0} - cos {θ}_{0} ) * r$$
where PD represents the projection difference, θ₀ represents the polar angle in the polar coordinate data of the variety to be tested, θ₀ is equal to or larger than 0 degree (°) and less than 360°, and r represents the radius in the polar coordinate data of the variety to be tested.

2. The polar coordinate-based method for evaluating the nutrient efficiency of the crop as claimed in claim 1, wherein criteria for the evaluating a nutrient efficiency of the variety to be tested through the projection difference are as follows:
when PD<0, the nutrient efficiency of the variety to be tested is lower than that of the plurality of control varieties;
when PD=0, the nutrient efficiency of the variety to be tested is equal to that of the plurality of control varieties;
when PD>0, the nutrient efficiency of the variety to be tested is higher than that of the plurality of control varieties.

3. The polar coordinate-based method for evaluating the nutrient efficiency of the crop as claimed in claim 1, further comprising evaluating a yield potential of the variety to be tested by using the polar angle in the polar coordinate data of the variety to be tested as an index.

4. The polar coordinate-based method for evaluating the nutrient efficiency of the crop as claimed in claim 3, wherein criteria for the evaluating a yield potential of the variety to be tested by using the polar angle are as follows:
when 0<θ₀<180°, the yield potential of the variety to be tested is higher than that of the plurality of control varieties;
when 180°<θ₀<360°, the yield potential of the variety to be tested is lower than that of the plurality of control varieties.

5. The polar coordinate-based method for evaluating the nutrient efficiency of the crop as claimed in claim 1, further comprising comprehensively evaluating the nutrient efficiency and a yield potential of the variety to be tested through the projection difference and the polar angle in the polar coordinate data of the variety to be tested.

6. The polar coordinate-based method for evaluating the nutrient efficiency of the crop as claimed in claim 5, wherein criteria for the comprehensively evaluating the nutrient efficiency and a yield potential of the variety to be tested are as follows:
when PD>0 and 45°<θ₀<180°, the nutrient efficiency and the yield potential of the variety to be tested are both higher than those of the plurality of control varieties;
when PD>0 and 180°<θ₀<225°, the nutrient efficiency of the variety to be tested is higher than that of the plurality of the control varieties, but the yield potential of the variety to be tested is lower than that of the plurality of control varieties;
when PD<0 and 0<θ₀<45°, the nutrient efficiency of the variety to be tested is lower than that of the plurality of control varieties, but the yield potential of the variety to be tested is higher than that of the plurality of control varieties;
when PD<0 and 225°<θ₀<360°, the nutrient efficiency and the yield potential of the variety to be tested are both lower than those of the plurality of control varieties.

7. The polar coordinate-based method for evaluating the nutrient efficiency of the crop as claimed in claim 1, wherein the nutrient efficiency refers to a utilization efficiency of nitrogen, phosphorus, and potassium by the crop.

8. The polar coordinate-based method for evaluating the nutrient efficiency of the crop as claimed in claim 1, wherein the crop is selected from a group consisting of corn, wheat, rice, soybean, fruits, and vegetables.
